(19) United States
(12) Patent Application Publication
(10) Pub. No.: US 2015/0094608 A1
(43) Pub. Date: Apr. 2, 2015

(54) SYSTEMS AND METHODS OF NON-INVASIVELY DETERMINING INTERNAL TEMPERATURE

(71) Applicant: Brain Temp, Inc., Englewood, CO (US)

(72) Inventors: Robert C. Allison, Rancho Palos Verdes, CA (US); John McCarthy, Newbury, NH (US); Leroy D. Geist, Aurora, CO (US)

(21) Appl. No.: 14/497,293

(22) Filed: Sep. 25, 2014

Related U.S. Application Data
(60) Provisional application No. 61/883,990, filed on Sep. 28, 2013.

(51) Int. Cl. A61B 5/01; A61B 5/00

(52) U.S. Cl. CPC A61B 5/01; A61B 5/0008; A61B 5/742; A61B 5/4064; A61B 5/7278
USPC 600/549

(57) ABSTRACT

A system for and noninvasively monitoring temperature within a body of a subject includes a transducer and an interface unit. The transducer receives native temperature signals, which are electromagnetic signals. One or both of the transducer and the interface unit convert the native temperature signals to a standard temperature signal. The interface unit may be configured to be coupled to a standard temperature signal-receiving apparatus, such as a standard vital signs monitor or a standard body temperature management system, which may recognize the standard temperature signal. Methods for noninvasively monitoring temperature, including receiving native temperature signals and converting the native temperature signals to a standard temperature signal, are also disclosed.

### TECHNICAL FIELD

This disclosure relates generally to systems and methods for noninvasively monitoring temperature within a body of a subject and, more specifically, to systems and methods for noninvasively obtaining an accurate measure of brain temperature.

### SUMMARY

In one aspect, systems for noninvasively monitoring temperature within a body of a subject, or an internal temperature at a selected location within the subject's body, are disclosed. Such a system includes a transducer and an interface unit.

The transducer of a system for noninvasive temperature monitoring is configured to receive electromagnetic radiation that originates from tissues inside of the subject's body. At least some electromagnetic radiation that originates from tissues within a subject's body provides an indication of the temperature at the location from which that electromagnetic radiation originates. Accordingly, electromagnetic radiation that corresponds to a temperature within the body of a subject may also be referred to herein as a “native temperature signal.” In some embodiments, the transducer may be configured to be positioned against an externally accessible surface (e.g., on skin, etc.) of the subject's body to receive native temperature signals that originate from nearby tissue.

The interface unit of a system for noninvasive temperature monitoring is configured to process a native temperature signal received by the transducer in a manner that provides a standardized temperature signal, which is also referred to herein as a “standard temperature signal,” that may be recognized by a standard vital signs monitor (e.g., a bedside monitor, etc.), or patient monitor. In a specific embodiment, each standardized temperature signal may comprise a thermistor-type signal. Thus, the interface unit is configured to translate native temperature signals to standardized temperature signals. By outputting standardized temperature signals, the interface unit may enable communication between the transducer for noninvasive temperature monitoring and an apparatus, such as a standard vital signs monitor (e.g., a monitor for displaying parameters such as temperature, blood pressure, blood oxidation, heart rhythms, etc.), a standard body temperature management system (e.g., a clinical hypothermia system, etc.) or the like, that is configured to receive a temperature input in the form of a standardized temperature signal (e.g., a standard electrical resistance, etc.).

In some embodiments, conversion of a native temperature signal to a standardized temperature signal may involve consideration of a reliable reference temperature. The reference temperature may be obtained by a reference temperature sensor, which may comprise part of the transducer or part of the interface unit, or it may be an independent element. In a specific embodiment, the reference temperature sensor may be configured to obtain a reliable reference temperature signal from an externally accessible location of the subject's body, and the interface unit may receive the reference temperature signal and consider it in providing a corresponding standardized temperature signal.

A system for noninvasively monitoring temperature may also include a standard vital signs monitor, such as a bedside monitor, as well as a communication link between the interface unit and the standard vital signs monitor. The communication link enables standardized temperature signals to be communicated from the interface unit to the standard vital signs monitor. In a specific embodiment, the communication link may comprise a cable that includes a thermistor connector that will couple with a standard thermistor input of the standard vital signs monitor.

In a method for noninvasively monitoring an internal temperature within a subject's body, one or more native temperature signals that originate from tissue at a location within the subject's body that is not externally accessible may be received at a location that is readily accessible (e.g., without requiring surgery, catheterization, etc.) from outside of the subject's body. In addition, the native temperature signals may be processed to provide one or more standardized temperature signals. Each standardized temperature signal may be communicated to a standard vital signs monitor, which may then provide a user perceptible output of the temperature at a location of interest (e.g., the brain, etc.) within the subject's body.

Other aspects, as well as features and advantages of various aspects, of the disclosed subject matter will become apparent to those of ordinary skill in the art through consideration of the ensuing description, the accompanying drawings and the appended claims.

### BRIEF DESCRIPTION OF THE DRAWINGS

In the drawings,

FIG. 1 is a schematic representation of an embodiment of a system for noninvasively monitoring temperature at a location within a subject's body;

FIG. 2 depicts an embodiment of a transducer of the embodiment of system schematically represented by FIG. 1, as well as interaction between the transducer and a location of interest within a body of a subject;

FIG. 3 shows an embodiment of an interface unit of the embodiment of system schematically represented by FIG. 1; and

FIG. 4 is a flow chart illustrating an embodiment of the process flow that occurs as the processing element of an interface unit executes programming to convert an electrical representation of an electromagnetic native temperature signal to a standardized temperature signal that is compatible with a standard vital signs monitor.

### DETAILED DESCRIPTION

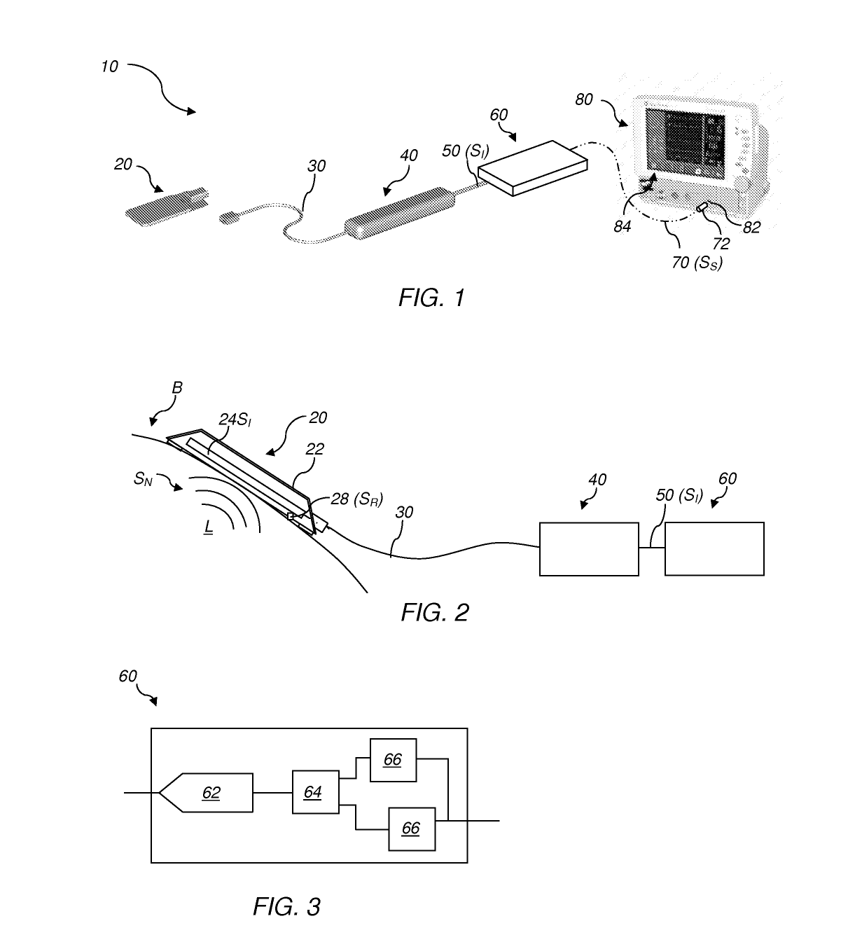

In FIG. 1, an embodiment of a system 10 for noninvasively measuring temperature within a body of a subject is illustrated. System 10 includes a transducer 20, a radiometer 40 and an interface unit 60, and optionally includes a temperature signal-receiving apparatus 80 that receives a standard temperature signal (e.g., a standard electrical resistance, etc.), such as a standard vital signs monitor (as depicted by FIG. 1), a body temperature management system (e.g., a clinical hypothermia system, etc.) or the like.

The transducer 20 is configured to receive electromagnetic radiation that originates from tissues inside of a subject's body B. As shown in FIG. 2, the transducer 20 includes a housing 22 that carries one or more antennas 24. The housing 22 may be configured to be positioned at a location where it will receive native temperature signals S_{N} originating from a location of interest L within the subject's body B. In some embodiments, the housing 22 may be configured to be placed against a surface of the subject's body B that may be readily accessed from outside of the subject's body B. In a specific embodiment, the housing 22 may be configured to position the transducer 20 adjacent to a portion of the subject's head.

When the transducer 20 is properly positioned relative to a location of interest L, each antenna 24 of the transducer 20 may receive a native temperature signal S_{N} originating from that location of interest L. In various embodiments, each antenna 24 may be configured to receive a native temperature signal S_{N} that comprise electromagnetic radiation in the so-called “microwave” portion of the electromagnetic spectrum. Without limitation, each antenna 24 may be configured to receive a native temperature signal S_{N} of a specific frequency, or native temperature signals S_{N} within a bandwidth of frequencies. In a specific embodiment, the range of frequencies of microwave radiation that may be received by the antenna 24 of the transducer 20 may include native temperature signals S_{N} having frequencies in the range of about 4 GHz±200 MHz. Specific embodiments of such a transducer 20 and antenna 24 are disclosed by PCT International Publication No. WO 2013/090047 A2 of Meridian Medical Systems, LLC, which was published on Jun. 20, 2013.

In addition to being configured to receive a native temperature signal S_{N}, each antenna 24 of the transducer 20 is configured to convert the native temperature signal S_{N}, which are electromagnetic, to a voltage, which is an electrical signal.

Optionally, the system 10 may include a reference temperature sensor 28, such as a thermistor, that provides a reliable (e.g., accurate, etc.) temperature measurement. In some embodiments, the reference temperature sensor 28 may be positioned to place the same into close proximity to, or even into contact with, a location from which a reference temperature measurement is to be obtained (e.g., skin, etc.). A reference temperature sensor 28 may be configured to generate a reference temperature signal S_{R} of a known type.

One or more cables 30 may extend from the transducer 20 to the radiometer 40. A cable 30 may enable the communication of voltages from the transducer 20 to the radiometer 40. In embodiments where the reference temperature sensor 28 is part of the transducer 20, a cable 30 may also be configured to convey one or more reference temperature signals S_{R} from the transducer 20.

The radiometer 40 of a system 10 for noninvasively measuring temperature within a body B of a subject (FIGS. 1 and 2) is configured to measure the voltages received from the transducer 20. The radiometer 40 converts the voltages to intermediate signals S_{I}, which are then conveyed to the interface unit 60. In some embodiments, a radiometer 40 may function in conjunction with one or more other components that improve the quality of the intermediate signals S_{I} produced thereby (e.g., amplify the incoming signal; reduce, filter, or eliminate noise, etc.; etc.).

In the depicted embodiment, the intermediate signals S_{I} are conveyed from the radiometer 40 to a separate interface unit 60 by way of a cable 50 that electrically couples the interface unit 60 to the radiometer 40. Alternatively, the radiometer 40 may comprise a part of an interface unit (e.g., the radiometer 40 may be housed with components of an interface unit 60, such as the components that are depicted by and described with reference to FIG. 3, etc.).

The interface unit 60 is configured to convert, or translate, each intermediate signal S_{I} received from the radiometer 40 and, ultimately, each native temperature signal S_{N} received by an antenna 24 of the transducer 20, to a standardized temperature signal S_{S}. Turning now to FIG. 3, in a specific embodiment, the interface unit 60 includes a signal converter 62 (e.g., an analog-to-digital (A/D) converter, etc.), a processing element 64 and one or more standardization converters 66.

The converter 62 of the interface unit 60 receives each intermediate signal S_{I} from the radiometer 40 and converts that signal to a signal type that may be processed by other components of the interface unit 60. In embodiments where the converter 62 comprises an A/D converter, it may convert a voltage to a digital temperature signal. In addition, in embodiments where the system 10 includes a reference temperature sensor 28, the converter 62 may receive each reference temperature signal S_{R} and convert it to a reference signal of a desired signal type (e.g., in embodiments where the converter 62 comprises an A/D converter, to a digital reference signal, etc.).

The temperature signals and any reference signals (which are collectively referred to herein as “converted signals”) output by the converter 62 may be received and processed by the processing element 64. The processing element 64 may be programmed, based on the digital signals it receives, to calculate and output a composite temperature signal.

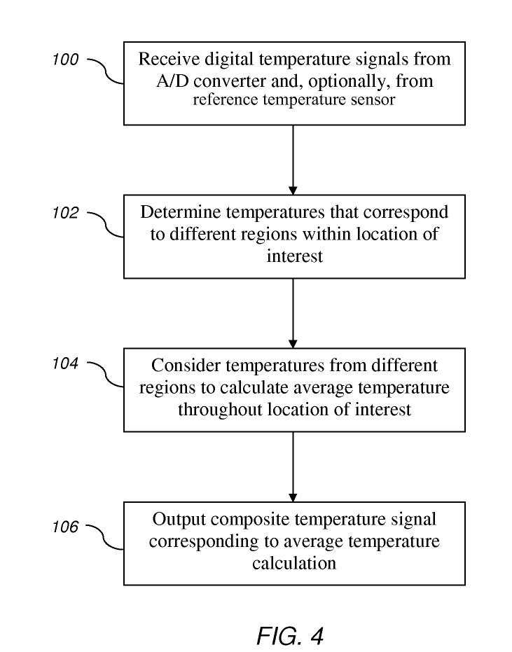

With added reference to FIG. 4, an embodiment of the process flow, as controlled by programming executed by a processing element 64 of the interface unit 60, is depicted. At reference 100 of FIG. 4, the processing element 64 receives converted signals from the converter 62 and, optionally, from a reference temperature sensor 28. At reference 102, the processing element 64 uses the native temperature signals S_{N}, from the different regions within the location of interest L to calculate a composite (e.g., average, weighted average, etc.) temperature throughout the location of interest L. At reference 104 of FIG. 4, the processing element 64 determines temperatures that correspond to one or more regions (e.g., depths, etc.) within the location of interest L (e.g., a volume of tissue, etc.) in the body B of a subject (FIG. 2). At reference 106, the processing element 64 outputs a composite temperature signal that corresponds to one or more specific locations of interest L based on the composite temperature calculation made at reference 104.

When the processing element 64 outputs a composite temperature signal, that signal is a digital signal. The processing element 64 transmits the composite temperature signal to the one or more standardization converters 66. Each standardization converter 66 converts the composite temperature signal to a standardized temperature signal S_{S} (FIG. 1). The standardized temperature signal S_{S} may comprise an analog signal, such as a standard resistance, that resembles a temperature signal generated by a conventional thermistor; i.e., the standardized temperature signal S_{S} may be a thermistor-type signal. Thus, the standardized temperature signal S_{S} may be recognizable to a standard vital signs monitor (FIG. 1). In some embodiments, each standardization converter 66 may comprise a digital potentiometer.

With returned reference to FIG. 1, the system 10 may include a cable 70 for establishing communication between the interface unit 60 and a temperature signal-receiving apparatus 80 that receives a standard temperature signal, such as a standard vital signs monitor, a body temperature management system or the like. The cable 70 may include a connector 72 at one end thereof. In embodiments where the interface unit 60 outputs a standardized temperature signal S_{S} that resembles the output of a thermistor, the connector 72 at the end of the cable 70 may comprise a standard thermistor-type connector. The connector 72 may be configured to couple with a temperature input 82 (e.g., an input for a thermistor type connector, etc.) of the temperature signal-receiving apparatus 80. In a specific, but non-limiting embodiment, the temperature input 82 may comprise an input for a YSI 400-series or similar thermistor; thus, the connector 72 of the cable 70 may comprise a complementary YSI 400-series or similar thermistor connector. Thus, the cable 70 and its connector 72 may enable communication of the standardized temperature signal S_{S} from the interface unit 60 to the temperature signal-receiving apparatus 80 through the temperature input 82. Since the standardized temperature signal S_{S} is received by the temperature signal-receiving apparatus 80 through the temperature input 82, the temperature signal-receiving apparatus 80 may recognize and use the S_{S}. As an example, in embodiments where the temperature signal-receiving apparatus 80 comprises a standard vital signs monitor, a display element of the temperature signal-receiving apparatus 80 with a dedicated temperature display element 84 may be configured to display a temperature that corresponds to the standardized temperature signal S_{S}.

Various embodiments of standard vital signs monitors that may be employed as the temperature signal-receiving apparatus 80 of a system 10 for non-invasively monitoring temperature within the body B of a subject include, but are not limited to, the S/5™ series vital signs monitors available from the Datex-Ohmeda division of GE Healthcare. Some non-limiting examples of body temperature management systems that may be employed as the temperature signal-receiving apparatus 80 of a system 10 include the ARCTIC SUN® 5000 temperature management system available from Medivance, Inc., of Louisville, Colo.; the THERMOGARD XP® thermal regulation system available from Zoll Medical Corporation of Chelmsford, Mass.; and the InnerCool RTx endovascular system available from Philips Healthcare of Best, the Netherlands.

With returned reference to FIG. 2, a method for noninvasively monitoring an internal temperature within a subject's body B may include monitoring for one or more native temperature signals S_{N} that originate from within a subject's body B. Such monitoring may be conducted, or effected, with a transducer 20 positioned outside of the subject's body B. The transducer 20 may be positioned at a location from which native temperature signals S_{N} from a location of interest L within the subject's body B are likely to be detected. In the embodiment illustrated by FIG. 2, the transducer 20 is positioned against the subject's head, near a portion of the subject's skull, to facilitate monitoring of a temperature of a portion of the subject's brain.

As FIG. 2 depicts, native temperature signals S_{N} radiate from a variety of locations throughout a region at the location of interest L. As the temperature may vary at different locations within the location of interest L, temperatures throughout the location of interest L may be considered in determining a composite (e.g., average, weighted average, etc.) temperature throughout the location of interest L.

In the specific, but nonlimiting embodiment depicted by FIG. 2, the location of interest L may include a portion of a subject's head. Without limitation, the location of interest L may extend from an outer boundary at the outer surface of skin on the subject's head, or the scalp, to an inner location beneath the surface of the skin on the subject's head. In some embodiments, the outer boundary may comprise the surface of the skin. In other embodiments, the outer boundary may be located at or directly adjacent to an inner surface of the subject's skull, thus omitting the skin and the skull. The inner location may be located at least 5 mm beneath the outer surface of skin on the subject's head (i.e., at least 5 mm deep), at least 1 cm deep, at least 1.5 cm deep, about 2 cm deep or at least 2 cm deep. The frequency of a native temperature signal S_{N} may correspond to the depth from which that native temperature signal S_{N} originated, with lower frequency signals corresponding to greater depths of origination. Thus, the location of interest L may be defined by selecting native temperature signals S_{N} within a predetermined range of frequencies (e.g., by configuration of the antenna(s), with filters, etc., or any combination of the foregoing).

Once native temperature signals S_{N} have been detected, they may be processed in accordance with the teachings of this disclosure to generate a standardized temperature signal S_{S}, and transmitted to a temperature signal-receiving apparatus 80, such as a standard vital signs monitor (FIG. 1), which may display a temperature value that corresponds to the standardized temperature signal S_{S}, or a body temperature management system, which used the standardized temperature signal S_{S} as a basis for actions that may alter or maintain the temperature of a portion of a subject's body, as well as optionally display a temperature value that corresponds to the standardized temperature signal S_{S}.

Although the foregoing description sets forth many specifics, these should not be construed as limiting the scope of any of the claims, but merely as providing illustrations of some embodiments and variations of elements or features of the disclosed subject matter. Other embodiments of the disclosed subject matter may be devised which do not depart from the spirit or scope of any of the claims. Features from different embodiments may be employed in combination. Accordingly, the scope of each claim is limited only by its plain language and the legal equivalents thereto.

1. A system for noninvasively measuring a temperature within a body of a subject, comprising:
a transducer for receiving emissions indicative of temperature from one or more locations in a volume of tissue within a body of a subject;
a reference temperature sensor for determining a temperature on skin of the subject adjacent to the volume of tissue and providing a reference temperature signal;
a radiometer for receiving one or more temperature signals from the transducer and for converting each temperature signal to a corresponding voltage; and
an interface unit, including:
at least one converter for converting each voltage to a converted temperature signal and for converting the reference temperature signal to a converted temperature signal;
a processing element for receiving and processing each converted temperature signal from the converter to calculate a composite temperature signal; and
a standardization converter for receiving the composite temperature signal and outputting a corresponding, standard temperature signal recognizable by a standard temperature signal-receiving apparatus.

2. The system of claim 1, wherein the transducer includes at least one microwave antenna.

3. The system of claim 1, wherein the composite temperature signal comprises a weighted average temperature throughout the volume of tissue.

4. The system of claim 3, wherein the volume of tissue comprises a volume of brain tissue.

5. The system of claim 4, wherein the volume of brain tissue is defined at least in part by an outer surface of a scalp of the individual and a predetermined depth beneath the outer surface of the scalp.

6. The system of claim 5, wherein the predetermined depth is at least 5 mm.

7. The system of claim 6, wherein the predetermined depth is at least 1 cm.

8. The system of claim 7, wherein the predetermined depth is about 2 cm.

9. The system of claim 8, wherein the predetermined depth is at least 2 cm.

10. The system of claim 1, further comprising:
a cable for communicating the standard temperature signal from the converter to the standard temperature signal-receiving apparatus, the cable including an end configured to be coupled with a temperature input of the standard temperature signal-receiving apparatus.

11. The system of claim 10, wherein the standard temperature signal comprises a thermistor-type signal and the cable is configured to convey the thermistor-type signal to the standard temperature signal-receiving apparatus.

12. The system of claim 1, wherein the radiometer is part of the interface unit.

13. A system for noninvasively measuring a temperature within a body of a subject, comprising:
a transducer for receiving emissions indicative of temperature from a plurality of locations in a volume of tissue within a body of a subject;
a reference temperature sensor for determining a temperature on skin of the subject adjacent to the volume of tissue and providing a reference temperature signal;
a radiometer for receiving a plurality of temperature signals from the transducer and for converting each temperature signal of the plurality to a corresponding voltage;
an interface unit, including:
at least one converter for converting each voltage to a converted temperature signal and for converting the reference temperature signal to a converted temperature signal; and
a processing element for receiving and processing each converted temperature signal from the converter to calculate a composite temperature signal corresponding to a weighted average temperature across the volume of temperature and to output a composite temperature signal.

14. The system of claim 13, wherein the transducer includes at least one microwave antenna.

15. The system of claim 13, wherein the composite temperature signal comprises an average temperature throughout the volume of tissue.

16. The system of claim 15, wherein the volume of tissue comprises a volume of brain tissue.

17. The system of claim 16, wherein the volume of brain tissue is defined at least in part by an outer surface of a scalp of the individual and a predetermined depth beneath the outer surface of the scalp.

18. The system of claim 17, wherein the predetermined depth is at least 5 mm.

19. The system of claim 18, wherein the predetermined depth is at least 1 cm.

20. The system of claim 19, wherein the predetermined depth is about 2 cm.

21. The system of claim 19, wherein the predetermined depth is at least 2 cm.

22. The system of claim 13, further comprising:
a cable for communicating the standard temperature signal from the converter to a standard temperature signal-receiving apparatus, the cable including an end configured to be coupled with a temperature input of the standard temperature signal-receiving apparatus.

23. The system of claim 22, wherein the standard temperature signal comprises a thermistor-type signal and the cable is configured to convey the thermistor-type signal to the standard temperature signal-receiving apparatus.

24. The system of claim 1, wherein the radiometer is part of the interface unit.

25. A system for non-invasively measuring a temperature within a body, comprising:
a transducer for receiving:
internal temperature signals generated within a body of a subject from a location on the body of the subject; and
a reference temperature signal from an externally accessible location of the body; and
an interface unit for converting internal temperature signals and the reference temperature signal to a standard temperature signal readable by a standard vital signs monitor.

26. The system of claim 25, further comprising:
a cable for communicating the standard temperature signal from the interface unit to the standard vital signs monitor, the cable including an end configured to be coupled with a temperature input port of the standard vital signs monitor.

27. The system of claim 26, further comprising:
the standard vital signs monitor.

28. The system of claim 25, further comprising:
a radiometer between the transducer and the interface unit.

29. An interface unit for establishing communication between a transducer for noninvasively monitoring temperature within a body of a subject and a standard temperature signal-receiving apparatus, the interface unit comprising:
a converter for converting each voltage to a converted temperature signal and for converting the reference temperature signal to a converted reference temperature signal;
a processing element for receiving and processing each converted temperature signal from the converter to calculate a composite temperature signal corresponding to a weighted average temperature across the volume of temperature and to output a composite temperature signal; and
a standardization converter for receiving the composite temperature signal and outputting a corresponding, standard temperature signal recognizable by a standard temperature signal-receiving apparatus.

30. The interface unit of claim 29, further comprising:
a radiometer for receiving a plurality of temperature signals from a transducer and for converting each temperature signal of the plurality to a corresponding voltage.

31. The interface unit of claim 30, wherein the standardization converter comprises at least one digital potentiometer.

32. A method for noninvasively monitoring a temperature within a body of a subject, comprising:
from a location outside of a body of a subject, receiving a native temperature signal originating from within the body of the subject; and
converting the native temperature signal to a standard temperature signal recognizable by a standard temperature signal-receiving apparatus.

33. The method of claim 32, wherein converting comprises generating a compound standard temperature signal from native temperature signals.

34. The method of claim 32, further comprising:
transmitting the native temperature signal to the standard temperature signal-receiving apparatus; and
displaying a temperature corresponding to the native temperature signal.
